(19) United States
(12) Patent Application Publication
(10) Pub. No.: US 2011/0009976 A1
(43) Pub. Date: Jan. 13, 2011

(54) ELEMENTS OF A FEMORAL PROSTHESIS WITH A MODULAR NECK, TOOLING FOR IMPLANTING A FEMORAL PROSTHESIS AND METHOD OF IMPLANTATION

(75) Inventor: Patrick Cruchet, Miami Beach, FL (US)

Correspondence Address: ARENT FOX LLP, 1050 CONNECTICUT AVENUE, N.W., SUITE 400, WASHINGTON, DC, 20036, US

(73) Assignee: Ceram Concept LLC, Newark, DE (US)

(21) Appl. No.: 12/499,435

(22) Filed: Jul. 8, 2009

(51) Int. Cl. A61F 2/32; A61B 17/58

(52) U.S. Cl. 623/22.46; 606/99

(57) ABSTRACT

A modular assembly for a femoral prosthesis comprises a femoral prosthetic implant forming a spindle of elongate and tapered shape and a one-piece neck. The implant consists of a diaphyseal lower part and a metaphyseal upper part having a flat face delimited by an outer edge and in which a cavity is formed. The neck is formed of a tenon having a shape corresponding to the cavity, said tenon being fitted into the cavity along an insertion axis YY perpendicular to the flat face and a rod provided with an interface for fixing a cup that is to be inserted into the acetabulum of the hip bone. The rod is provided with a tongue which protrudes parallel to the flat face in a radial direction with respect to the insertion axis and directed towards the interior of the obtuse salient angle, at a distance from the flat face of between 0.3 mm and 1.5 mm measured along the insertion axis, the tongue having a free end located at a greater distance from the insertion axis than the part of the edge located opposite the tongue. The free end of the tongue overhangs somewhat with respect to the outer edge of the flat face of the femoral prosthetic implant, in such a way as to ensure that, in the event of sinking of the implant into the medullar cavity, the tongue will come into abutment against the femoral calcar.

### TECHNICAL FIELD OF THE INVENTION

The invention relates to the field of femoral prostheses to be implanted in the human body.

### BACKGROUND OF THE INVENTION

As is well-known in the art, a hip prosthesis usually comprises a femoral prosthetic implant intended to be introduced into the medullary cavity of the femur and a neck designed to receive, directly or otherwise, a cup intended to cooperate with the acetabulum of the hip bone.

Essentially three types of prosthesis are known, namely a one-piece type in which the neck and the prosthetic head are integral with the femoral prosthetic implant, a two-piece type having wherein the neck is integral with the femoral prosthetic implant and cooperates with a modular head, and a three-piece type wherein each of the femoral prosthetic implant, neck and prosthetic head is modular. This latter modular design makes it possible to choose, during the implantation operation and according to the anatomy of the patient, firstly the femoral prosthetic implant, which best corresponds to the dimensions of the medullary cavity, then in a second step a neck which has the length and cervico-diaphyseal angle suitable for the patient, and finally a prosthetic head having the dimensions of the acetabulum.

It sometimes happens that, once the patient has regained its mobility and before bone rehabitation has taken place, the femoral prosthetic implant of the prosthesis sinks into the medullary cavity as a result of the considerable forces transmitted by the femur and the prosthesis.

To prevent such sinking, some prostheses of the two-piece type are provided with a bulge or collar that comes to rest on the femoral calcar and forms a stop. Examples of such stops are illustrated in the documents U.S. Pat. No. 5,019,108; U.S. Pat. No. 6,695,884; U.S. Pat. No. 4,536,894; U.S. Pat. No. 4,520,511; U.S. Pat. No. 5,035,717; U.S. Pat. No. 4,881,536; U.S. Pat. No. 4,642,124; FR 2 242 065; FR 2 667 785; FR 2 784 576 and WO 94/07438.

Such an arrangement cannot be transposed as such to prostheses with a modular neck since, from a mechanical point of view, it is not possible to achieve simultaneously without cement the fixing of the neck to the femoral prosthetic implant and the coming into abutment of the tongue against the femoral section.

### SUMMARY OF THE INVENTION

The invention aims to remedy the disadvantages of the prior art and in particular to provide means to prevent any sinking into the medullary cavity of the spindle of a prosthesis with a modular neck, in particular when no cements are used.

To this end, there is proposed according to a first aspect of the invention a one-piece neck for a modular femoral prosthesis, comprising:
a tenon for fitting the one-piece neck into a correspondingly shaped cavity of a femoral prosthetic implant, the tenon protruding along an axis of insertion of the tenon into the cavity of the prosthetic implant; and
a rod provided with an interface for fixing a cup that is to be inserted into the acetabulum of the hip bone, the rod being provided with a tongue which protrudes radially with respect to the insertion axis and with respect to the tenon, the tongue being axially at a distance and set back from the tenon.

Due to its positioning on the rod set back from the tenon, the tongue will not be in contact with the bone or with the femoral prosthetic implant when the tenon is inserted into the cavity of the prosthetic implant acting as a mortise. The tongue will therefore not hinder the fitting of the tenon into the cavity. If the femoral implant sinks into the medullary cavity, the part of the tongue protruding radially beyond the tenon comes into abutment with the bone to prevent it from further sinking.

Preferably, the tongue has a face turned axially towards the tenon, having a surface area greater than 0.5 cm². Furthermore, the tongue preferably has a length, measured in a radial direction perpendicular to the insertion axis, of more than 3 mm and preferably more than 10 mm. These dimensions are given as an indication. In any case they are of such a nature as to allow a sufficient part of the tongue to come to bear against the edge of the bone or of the femoral calcar.

Preferably, the tenon forms a Morse taper. Here, a Morse taper or cone is intended to mean a frustoconical geometric shape having a conicity of around 5%. The base of the cone here is preferably an oblong shape.

The neck is preferably made of metal, e.g. titanium, titanium alloy or a chrome-cobalt alloy.

According to another aspect of the invention, the latter relates to a modular assembly for a femoral prosthesis, comprising:
a femoral prosthetic implant forming a spindle of elongate and tapered shape, defined by a first longitudinal axis XX and consisting of a diaphyseal lower part and a metaphyseal upper part having a flat face delimited by an outer edge and in which a cavity is formed,
a one-piece neck formed of
   a tenon having a shape corresponding to the cavity, said tenon being fitted into the cavity along an insertion axis YY perpendicular to the flat face and defining with the axis XX an obtuse salient angle, and
   a rod provided with an interface for fixing a cup that is to be inserted into the acetabulum of the hip bone, the rod being provided with a tongue which protrudes parallel to the flat face in a radial direction with respect to the insertion axis and directed towards the interior of the obtuse salient angle, at a distance from the flat face of between 0.3 mm and 1.5 mm measured along the insertion axis, the tongue having a free end located at a greater distance from the insertion axis than the part of the edge located opposite the tongue.

The free end of the tongue overhangs somewhat with respect to the outer edge of the flat face of the femoral prosthetic implant, in such a way as to ensure that, in the event of sinking of the implant, the tongue will come into abutment against the femoral calcar. Preferably, the part of the edge located opposite the tongue is located at a distance L0 from the insertion axis, and the free end of the tongue is located at a distance L such that L-L0>2 mm, and preferably such that 3 mm<L-L0<20 mm.

Preferably, the free end of the tongue has a face turned towards the femoral prosthetic implant and having a surface area of between 0.5 and 1.5 cm².

The tenon preferably forms a Morse cone, which fits and is wedged into the cavity of the femoral prosthetic implant. The base of the cone may be oblong or more generally oval, and may have a major axis oriented in the radial direction of the tongue.

According to another aspect of the invention, the latter furthermore relates to a tooling for installing a modular sub-assembly for a femoral prosthesis, comprising:
a reamer comprising a sleeve and a head, the head having:
   a flat bearing face,
   a cylindrical cavity opening onto the bearing face and defining an axis of rotation of the reamer perpendicular to the bearing face, and
   a toothed sector comprising at least one tooth provided with a cutting edge which protrudes axially with respect to the bearing face, at a distance from the axis of rotation and from the cylindrical cavity, at least part of the bearing surface being located, relative to the cylindrical cavity, diametrically opposite the toothed sector.

The tooling is intended for milling the calcar over a small thickness, before the neck is installed, so as to ensure that no contact between the calcar and the tongue will take place during the installation of the neck.

Preferably, the axial distance between the cutting edge and the bearing face is between 1 and 3 mm, preferably around 1.5 mm. This distance defines the thickness of calcar that will be milled by the tool.

In order to guide the reamer, the tooling furthermore preferably comprises a guide part having a tenon and a cylindrical rod cooperating with the cylindrical cavity so as to allow a rotation of the reamer about the axis of rotation. The tenon of the guide part is inserted into the cavity of the femoral prosthetic implant, the cylindrical rod defining the axis of rotation of the reamer.

The guide part may be made from a chirurgical plastic, for example a polytetrafluoroethylene.

According to another aspect of the invention, the latter relates to a method for implanting the femoral prosthesis described above using the tooling described above, comprising at least the following successive steps:
bone resection of the femoral neck;
opening and preparation of a cavity for receiving the femoral implant in the medullary cavity using a suitable file;
implantation of the femoral prosthetic implant until it is wedged in the bone cavity;
fitting of the guide part;
fitting of the reamer onto the guide part;
rotation of the reamer so as to mill away the femoral calcar over a medial or posteromedial angular sector;
determination of the length and angle of the neck using a modular test neck;
implantation of the modular neck so that the tongue is located at a minimum distance of 0.5 mm from the prosthetic implant and at least partially facing the milled angular sector of the calcar.

### BRIEF DESCRIPTION OF THE FIGURES

Other features and advantages of the invention will become apparent from reading the following description, with reference to the following figures, in which:

FIG. 1 shows a cross-section of a femoral implant having a modular neck and implanted in the acetabulum of a femur;

FIGS. 2A and 2B show two perspective views of the modular neck of FIG. 1;

FIG. 3 shows a cross-sectional view of a tooling for preparing the femur, after implantation of the femoral prosthetic implant and before insertion of the modular neck;

FIGS. 4A and 4B show schematic illustrations in a front view and profile view of a head of a reamer of the tooling of FIG. 3;

FIGS. 5A and 5B show schematic illustrations in a front and side view of a guide of the tooling of FIG. 3.

For greater clarity, identical or similar elements bear identical reference signs in the figures and in the following description.

### DETAILED DESCRIPTION OF AN EMBODIMENT

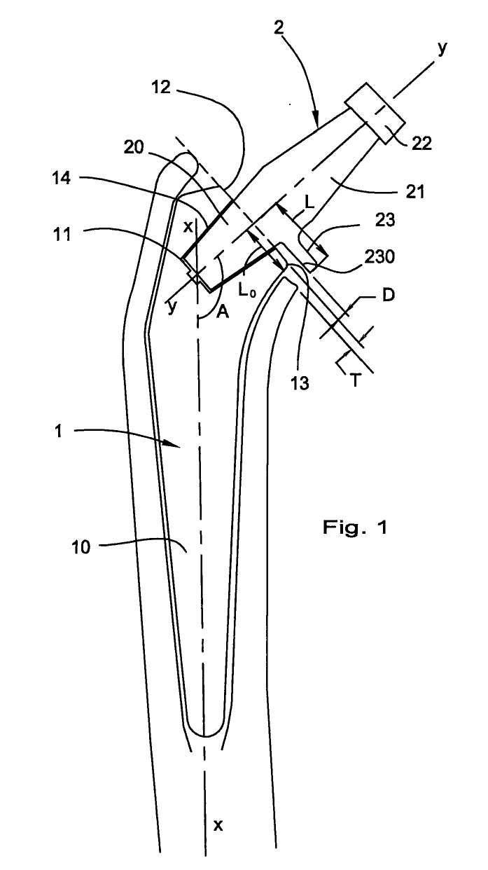

With reference to FIG. 1, the diaphysis of a femur has been separated from the femoral neck by sectioning using an oscillating saw along a bone section plane P, then the diaphyseal cavity was partially hollowed out and scraped using a scraper in order to introduce therein a prosthetic implant 1. The implant 1 has a diaphyseal part 10 of tapered shape along a longitudinal axis X-X which, during the implantation, becomes coincident with the femoral diaphyseal axis. The implant 10 widens in its metaphyseal upper part 11 and has a flat face 12 delimited by an outer edge 13 and in which there is formed a frustoconical cavity 14 defining an axis Y-Y, this axis being perpendicular to the flat face 12 and close to the diaphyseal axis of the head of the femur before sectioning.

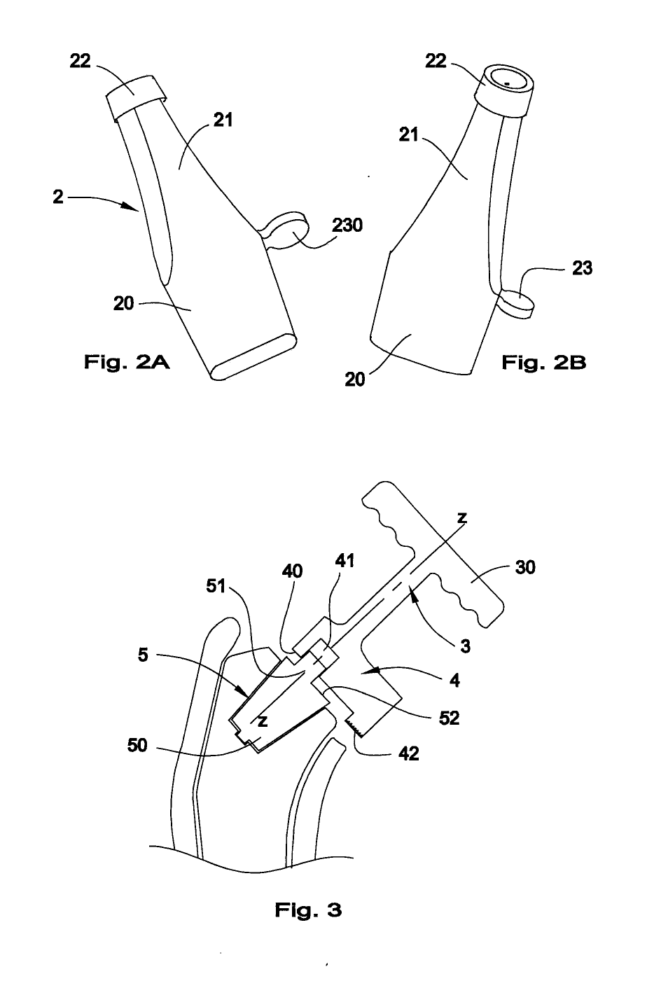

Into the frustoconical cavity 14 of the femoral prosthetic implant there has been fitted a correspondingly shaped tenon or cone 20 which constitutes the lower part of a modular neck 2 that provided with a protruding rod 21 which is provided at its end, in a manner known per se, with an interface 22 for fixing a cup (not shown) intended to be inserted into the acetabulum of the hip bone. In the example of embodiment, the cone 20 has an oblong base, as illustrated in FIGS. 2A and 2B. The cone 20 is a Morse cone, the small angle of opening making it easier to fit and wedge said cone into the corresponding frustoconical cavity 14 of the femoral prosthetic implant, without requiring any cement. The rod may be strictly in the axis Y-Y or may have a larger or smaller angle relative to this axis, for example varying between −15° and +15°, so as to recreate the actual diaphyseal angle of the femur of the patient. Similarly, the length of the rod may vary. It will be understood that the surgical team will have during the operation a range of available modular necks compatible with the prosthetic implant, and will be able to choose the most suitable neck during the operation, even after insertion of the femoral prosthetic implant.

According to the invention, the rod 21 is provided with a tongue 23 which protrudes radially with respect to the insertion axis YY and with respect to the tenon 20. As can be seen in FIG. 1, the tongue 23 is axially at a distance and set back from the plane P and from the tenon 20, the latter being housed entirely in the interior of the frustoconical cavity 14 of the femoral prosthetic implant 1 on the same side of the plane P as the femoral prosthetic implant 1.

The tongue 23 extends parallel to the flat face 12 towards the inside of an obtuse salient angle A (i.e. an angle such that 90°<A<180°) defined by the axis XX and the axis YY, and therefore towards the inside of the thigh of the patient. An axial clearance D measured along the insertion axis YY of preferably between 0.3 mm and 1.5 mm is maintained with the plane P. The tongue has a free end which protrudes radially beyond the edge of the flat face and is thus located directly opposite and at a distance from the femoral calcar, this overhanging part having a length, measured radially with respect to the axis YY, of preferably more than 2 mm and, in practice, of preferably between 3 and 20 mm. This length is the difference between the distance L of the end of the tongue 23 and the axis YY and the distance L0 between the edge 13 of the flat face 12 of the implant 1 and the axis YY. The face 230 of the free end of the tongue 23 which is turned towards the femoral prosthetic implant and towards the femoral calcar and is facing the bone has a surface area of preferably between 0.5 and 1.5 cm², and more generally a surface area that is sufficient for the tongue to be able to form a stop in the event of involuntary sinking of the femoral prosthetic implant into the diaphyseal cavity.

Remarkably the calcar, facing the tongue 23, is set back relative to the bone section plane P. This is because in a preparation phase, and preferably in an operating phase after the installation of the femoral prosthetic implant 1 and before the installation of the modular neck 2, the calcar has been hollowed out, preferably using a dedicated tooling which will be described below, in a narrow angular sector corresponding to that provided for the tongue 23. The bone material is hollowed out over a small thickness T, for example between 0.5 and 2 mm, this thickness being sufficient to ensure a lack of contact between the bone and the tongue 23 during the installation of the modular neck 2.

The tooling proposed for preparing the bone is shown in FIGS. 3 to 5B. It is composed of a reamer 3 and a guide accessory 5.

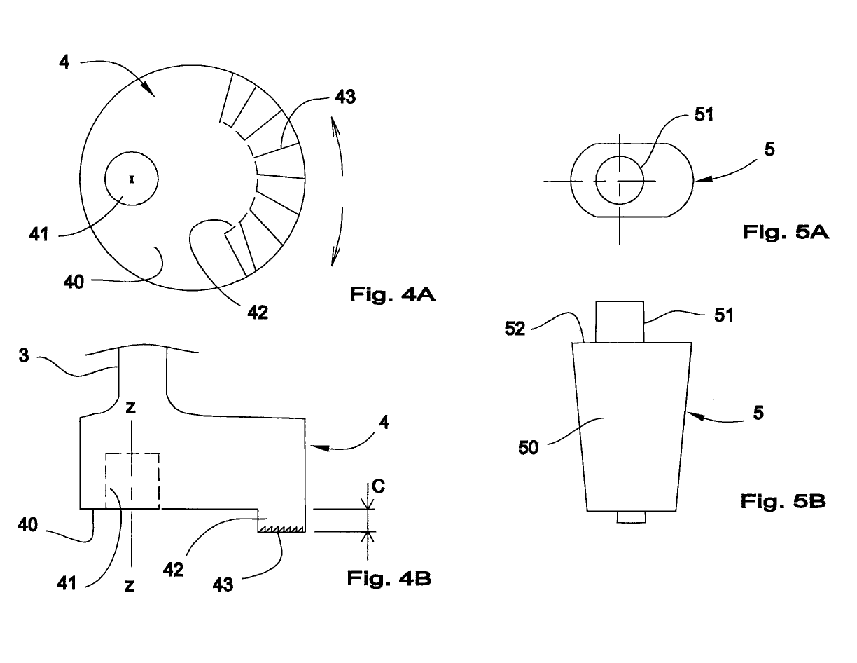

The reamer 3 comprises a handle 30, and a tool head 4 which has a flat bearing face 40, a cylindrical cavity 41 opening onto the bearing face 40 and defining an axis of rotation ZZ of the reamer perpendicular to the bearing face 40, and a toothed sector 42 comprising at least one tooth 43 having a cutting edge which protrudes axially with respect to the bearing face 40, at a distance from the axis of rotation ZZ and from the cylindrical cavity 41, at least part of the bearing face 40 being located, relative to the cylindrical cavity 41, diametrically opposite the toothed sector 42. As shown on FIG. 4B the axial distance C between the cutting edge and the bearing face 40 is between 1 and 3 mm, preferably around 1.5 mm.

The guide part, preferably made from chirurgical plastic, for example from polytetrafluoroethylene, has a tenon 50 and a cylindrical rod 51 cooperating with the cylindrical cavity to allow a rotation of the reamer about the axis of rotation ZZ.

The tooling is used as follows. After the implantation of the femoral prosthetic implant 1, the tenon 50 of the guide part is placed into the frustoconical cavity 14 of the femoral prosthetic implant. The height of the tenon is preferably such that when it is fully inserted in the cavity 14, the upper face 52 of the tenon 50 slightly protudes from the plane P, in practice by less than 1 mm. The cylindrical rod 51 of the guide part then protrudes perpendicular to the bone section plane P and defines an axis of rotation ZZ. This guide rod is fitted into the corresponding cylindrical cavity 41 of the reamer. The material and the dimensions of the guide part are chosen so as to allow the rod 51 to form a guide in translation and in rotation in the cylindrical cavity 41 of the reamer. The teeth 43 initially come into contact with the calcar. By virtue of rotations of the reamer back and forth about the axis of rotation defined by the rod, over a small medial or posteromedial angular sector of around 30 to 60°, the teeth 43 progressively hollow out the calcar until the bearing surface 40 bears against the upper face 52 of the tenon, in the section plane P. At this moment in time it is ensured that, in the milled zone, the calcar is set back from the plane P over a predetermined depth corresponding to the size of the teeth minus the protruding height of the tenon 50. In practice this depth is around 1 to 3 mm and preferably around 1.5 mm, measured in a direction perpendicular to the plane P.

Once this preparation has been carried out, the reamer 3 and the guide part 5 are removed and a test neck (not shown), designed to determine with precision the dimensions of the definitive neck, is inserted into the frustoconical cavity 14 of the femoral prosthetic implant 1. This part of the operating procedure is known per se and will not be precisely described here. Once the dimensions of the ideal modular neck have been determined, the neck suitable for the situation is selected from the range of available necks, and this modular neck 2 is fitted into the frustoconical cavity 1. It should be emphasised that of all the tenons successively introduced into the frustoconical cavity 14, namely that of the guide part 50, then that of the test neck and finally that of the definitive modular neck 2, only the latter has dimensions that favour the wedging thereof in the frustoconical cavity.

After the operation, once the patient has recovered its mobility, if for any reason the femoral prosthetic implant 1 starts sinking into the sinks into the medullary cavity, the tongue 23 will abut against the calcar and prevent further sinking.

Various modifications can be made without departing from the scope of the invention. In some cases, the removal of the calcar may be dispensed with, in particular if the plane defined by the flat upper face 12 of the implant 1 is at an angle with the bone section plane P.

The axis of the handle 30 of the reamer may be coaxial with the rotation axis ZZ or off center. The femoral prosthetic implant and the modular neck may be made from titanium. Some types of stainless steel and some chromium-cobalt alloys could also be used as an alternative.

What is claimed is:

1. A one-piece neck for a modular femoral prosthesis, comprising:
a tenon for fitting the one-piece neck into a correspondingly shaped cavity of a femoral prosthetic implant, the tenon protruding along an axis of insertion of the tenon into the cavity of the prosthetic implant; and
a rod provided with an interface for fixing a cup that is to be inserted into the acetabulum of the hip bone, the rod being provided with a tongue which protrudes radially with respect to the insertion axis and with respect to the tenon, the tongue being axially at a distance and set back from the tenon.

2. The neck according to claim 1, in which the tongue has a face turned axially towards the tenon, having a surface area of between 0.5 and 1.5 cm².

3. The neck according to claim 1, in which the tongue has a length, measured in a radial direction perpendicular to the insertion axis, of more than 3 mm and preferably more than 10 mm.

4. The neck according to claim 1, in which the tenon forms a Morse cone.

5. The neck according to claim 1, made from titanium or titanium alloy.

6. A modular assembly for a femoral prosthesis, comprising:
a femoral prosthetic implant forming a spindle of elongate and tapered shape, defined by a first longitudinal axis XX and consisting of a diaphyseal lower part and a metaphyseal upper part having a flat face delimited by an outer edge and in which a cavity is formed,
a one-piece neck formed of
a tenon having a shape corresponding to the cavity, said tenon being fitted into the cavity along an insertion axis YY perpendicular to the flat face and defining with the axis XX an obtuse salient angle, and
a rod provided with an interface for fixing a cup that is to be inserted into the acetabulum of the hip bone, the rod being provided with a tongue which protrudes parallel to the flat face in a radial direction with respect to the insertion axis and directed towards the interior of the obtuse salient angle, at a distance from the flat face of between 0.3 mm and 1.5 mm measured along the insertion axis, the tongue having a free end located at a greater distance from the insertion axis than the part of the edge located opposite the tongue.

7. The assembly according to claim 6, in which the part of the edge located opposite the tongue is located at a distance L0 from the insertion axis, and the free end of the tongue is located at a distance L such that L-L0>2 mm.

8. The assembly according to claim 6, in which the part of the edge located opposite the tongue is located at a distance L0 from the insertion axis, and the free end of the tongue is located at a distance L such that 3 mm<L-L0<20 mm.

9. The modular assembly according to claim 6, in which the free end of the tongue has a face turned towards the femoral prosthetic implant and having a surface area of between 0.5 and 1.5 cm².

10. The assembly according to claim 6, in which the tenon forms a Morse cone.

11. A tooling for installing a modular assembly for a femoral prosthesis, comprising:
a reamer comprising a sleeve and a head, the head having:
a flat bearing face,
a cylindrical cavity opening onto the bearing face and defining an axis of rotation of the reamer perpendicular to the bearing face, and
a toothed sector comprising at least one tooth provided with a cutting edge which protrudes axially with respect to the bearing face, at a distance from the axis of rotation and from the cylindrical cavity, at least part of the bearing surface being located, relative to the cylindrical cavity, diametrically opposite the toothed sector.

12. The tooling for installing a modular assembly for a femoral prosthesis according to claim 6, in which the axial distance between the cutting edge and the bearing face is between 1 and 3 mm, preferably around 1.5 mm.

13. The tooling for installing a modular assembly for a femoral prosthesis according to claim 6, furthermore comprising:
a guide part having a tenon and a cylindrical rod cooperating with the cylindrical cavity so as to allow a rotation of the reamer about the axis of rotation.

14. The tooling according to claim 11, in which the guide part is made from plastic.

15. The method for implanting a femoral prosthesis according to claim 6, using a tooling according to claim 11, comprising at least the following successive steps:
bone resection of the femoral neck;
opening and preparation of a cavity for receiving the femoral implant in the medullary cavity;
implantation of the femoral prosthetic implant until it is wedged in the bone cavity;
fitting of the guide part;
fitting of the reamer onto the guide part;
rotation of the reamer so as to mill away the femoral calcar over a medial or posteromedial angular sector;
determination of the length and angle of the neck;
implantation of the modular neck so that the tongue is located at a minimum distance of 0.5 mm from the prosthetic implant and at least partially facing the milled angular sector of the calcar.
